(19) United States
(12) Patent Application Publication
(10) Pub. No.: US 2015/0182574 A1
(43) Pub. Date: Jul. 2, 2015

(54) COMPOSITIONS AND METHODS FOR ENHANCING MOBILIZATION AND PROLIFERATION OF BLASTOMERE-LIKE STEM CELLS

(71) Applicant: Stemtech International, Inc., Pembroke Pines, FL (US)

(72) Inventor: Christian Drapeau, Austin, TX (US)

(73) Assignee: Stemtech International, Inc., Pembroke Pines, FL (US)

(21) Appl. No.: 14/408,845

(22) PCT Filed: Jul. 10, 2013
(86) PCT No.: PCT/US13/49897

§ 371 Date: Dec. 17, 2014

Related U.S. Application Data
(60) Provisional application No. 61/670,253, filed on Jul. 11, 2012.

(51) Int. Cl. A61K 36/704; A61K 35/748; A61K 31/737; C12N 5/0735

(52) U.S. Cl. CPC A61K 36/704; C12N 5/0606; A61K 35/748; A61K 31/737; C12N 2500/72

(57) ABSTRACT

The present invention provides a method of using mobilization agents to enhance stem cell trafficking in a subject, including very small embryonic-like (VSEL) stem cells. In one embodiment, a blended composition of algae, fruits, mushrooms, microorganisms, maternal fluids, and extracts thereof are used to promote trafficking of stem cells, resulting in migration of the stem cells to specific sites of maintenance and repair within tissues and/or organs.

### CROSS-REFERENCE TO RELATED APPLICATIONS

This application claims the benefit of priority to U.S. Provisional Patent Application Ser. No. 61/670,253 filed Jul. 11, 2012, which is incorporated by reference herein in its entirety.

### FIELD OF THE INVENTION

The present invention relates to the use of compositions and methods involved in regenerative processes in the body through stem cell mobilization, including very small embryonic like (VSEL) cells.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Stem cells (SC) are cells with the unique capacity to self-renew and to differentiate into various cell types of the body. Two well-known types of stem cells are embryonic stem cells and adult stem cells. Embryonic stem cells (ESCs) are extracted from 5-10 day old embryos and once isolated, can be grown in vitro and led to differentiate into virtually all of the cell types found in an adult organism. Adult stem cells (ASCs) are undifferentiated or primitive cells that can self-renew and differentiate into specialized cells of various tissues and are found in any living organism after birth. It is well-established that ASCs play key roles in the normal maintenance and regeneration processes of the body. For example, cells in the bone marrow and blood have long been known to replace and repair tissues in connection with routine homeostatic processes (e.g., formation of blood), as well as in response to injury (e.g., wound repair). (Drapeau, 2010).

However, increasing evidence clearly indicates a more expansive role for the capacity of ASCs in maintenance and regeneration. First, recent publications have made clear that ASCs are a wider group of cells than previously understood, and a much more highly heterogeneous population. (Ratajczak et al., 2008 and D'Ippolito et al., 2004). Importantly, this includes several key actors that were almost entirely unknown until recently. Second, many of these cells display remarkable features, such as wide plasticity (i.e., differentiation potential) and robust self-renewal capacity. Together, these combined categories of ASCs, including newly discovered actors, require a re-thinking of what has been understood to be the overall regeneration and repair capacity of an adult organism.

This shift in thinking focuses on the connection between healing and regeneration processes of the body, and the capacity of stem cells to differentiate into a broad variety of cell types. In this regard, stem cells have long been understood to serve as a resource for repair and replacement Classic adult stem cells such as bone marrow stem cells, and marrow stromal cells (MSCs), release from tissues of origin, circulate in a subject's circulatory or immune system, and migrate into various organs and tissues to become mature, terminally differentiated cells. Identification of new types of ASCs in the body, such as very small embryonic-like (VSELs), creates new opportunities to tap into further resources of the body for repair and regeneration. Importantly, enhancement of stem cell trafficking (i.e., release, circulation, homing and/or migration) can amplify these physiological processes and provide potential therapies for various pathologies. As various compositions and methods are known to motivate stem cell mobilization as a therapeutic approach, and it is of vital interest to understand how newly discovered types of ASCs can also participate in these processes.

Accordingly, the inventive compositions and methods disclosed herein enhance the release, circulation, homing and/or migration of stem cells within the body to promote healing and treatment of damaged tissues, as well as aid in the regeneration of tissues that suffer from some level of cellular loss, for greater vitality and reduced incidence of disease. In certain embodiments, mobilization agents are applied for newly discovered ASCs, such as very small embryonic-like (VSEL) cells.

### SUMMARY OF THE INVENTION

The following embodiments and aspects thereof described and illustrated in conjunction with compositions and methods are meant to be exemplary and illustrative, not limiting in scope.

Described herein is a method of increasing stem cell mobilization in a subject, including providing a mobilization agent capable of increasing stem cell mobilization, and administering a quantity of the mobilization agent to the subject in an amount sufficient to increase stem cell mobilization in the subject. In other embodiments, the mobilization agent is a composition including one or more of the following components selected from the group including: Aphanizomenon flos aquae or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma Lucidum or extracts thereof, and/or Cordyceps Sinensis or extracts thereof. In other embodiments, the mobilization agent is Aphanizomenon flos aquae or extracts thereof. In other embodiments, the mobilization agent is Polygonum multiflorum or extracts thereof. In other embodiments, the mobilization agent is fucoidan. In other embodiments, the stem cell is a very small embryonic-like (VSEL) stem cell. In other embodiments, the VSEL cell is an activated or quiescent VSEL. In other embodiments, the stem cell is a blastomere-like stem cell (BLSC) or epiblast-like stem cell (ELSC). In other embodiments, administering the quantity includes oral administration. In other embodiments, the oral administration includes use of a capsule or a pill.

Further described herein is a pharmaceutical composition including one or more of the following components selected from the group including: Aphanizomenon flos aquae or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma Lucidum or extracts thereof, and/or Cordyceps Sinensis or extracts thereof, and a pharmaceutically acceptable carrier.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 Cells on a hemacytometer stained with Trypan blue. Round, bright cells shown are activated very small embryonic-like (VSELs) stem cells, which are one example of blastomere-like stem cell (BLSCs). Darker cells in the background are quiescent VSELs. Cells were counted on a 16-square grid using a manual cell counter and diluted by a factor of 1:8. VSEL on border could be either a quiescent or activated VSEL depending on the focus.

FIG. 2 Samples of very small embryonic-like (VSELs) stem cells, which are one example of blastomere-like stem cell (BLSCs) are shown. After being incubated at room temperature and seeded 24 hours after blood draw. A) The undiluted sample on the day of seeding; B) The undiluted sample three days after seeding; C) The 1:10 dilution on the day of seeding; D) The 1:0 dilution three days after seeding; E) The 1:20 dilution on the day of seeding; F) The 1:10 dilution on the day of seeding.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russell, Molecular Cloning: A Laboratory Manual 3rd ed., Cold Spring Harbor Laboratory Press (Cold Spring Harbor, N.Y. 2001), Remington's Pharmaceutical Sciences, by E. W. Martin, Mack Publishing Co., Easton, Pa., 15th Edition (1975), describes compositions and formulations suitable for pharmaceutical delivery of the inventive compositions described herein provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods described herein. For purposes of the present invention, the following terms are defined below.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Aphanizomenon flos aquae” or “AFA” as used herein refers to a type of blue-green algae that is a freshwater species of cyanobacteria.

“Blue-green algae” as used herein refers to the common name for gram-negative photosynthetic bacteria belonging to division Cyanophyta that may exist in unicellular, colonial, or filamentous forms. Representative blue-green algae include, but are not limited to, Spirulina and Aphanizomenon species, one example being the Aphanizomenon flos aquae (AFA) species of blue-green algae. “Algae” is the plural form of “alga,” which is a cell of a microalgae species. For example, “blue green algae” refers to multiple cells of a single Aphanizomenon species, multiple cells of a single Spirulina species, or a mixture of cells from multiple Aphanizomenon and/or Spirulina species.

“Circulatory system” as used herein refers to the mechanisms for moving blood and blood components throughout the body of a subject, including the vascular and lymph systems. The mechanisms of the circulatory system include, but are not limited to, the heart, blood vessels (arteries, veins, and capillaries), and lymph vessels.

“Colostrum” as used herein refers to a fluid secreted by the mammary glands of female mammals during the first few days of lactation, containing various nutrients and protease inhibitors that keep it from being destroyed by the processes of digestion. Humans produce relatively small amounts of colostrum in the first two days after giving birth, but cows produce about nine gallons of colostrum. Colostrum contains concentrated levels of important immune modulators, including Transfer Factor, PRP, IGF-1, n-acetyl neuraminic acid, GMP, nucleic acid and defensins. Colostrum extracts have been shown to activate phagocytosis by monocytes and increase the reactive oxygen burst in polymorph nucleated cells. Colostrum was also shown to trigger natural killer (NK) cell activation and also trigger the secretion of anti-inflammatory cytokines in in vitro cell-based assays. References herein to colostrum also include derivatives and artificial substitutes thereof.

“Component of Polygonum multiflorum” as used herein refers to any fraction, extract, or isolated or purified molecule from Polygonum multiflorum. For example, the component is a protein or nucleic acid or a polysaccharide, a phytochemical, or a fraction of Polygonum multiflorum. Thus, in certain embodiments of the invention, components of Polygonum multiflorum are obtained by disrupting Polygonum multiflorum, adding an inorganic or organic solvent, and collecting fractions. Specific, non-limiting examples of fractions are isolated using high performance liquid chromatography, thin layer chromatography, or distillation. Fractionation may be based on the molecular weight or the hydrophobicity of the components of Polygonum multiflorum. Examples of components found in Polygonum multiflorum include hydroxyl stilbenes, anthraquinones and derivatives, lecithin, chrysophanic acid, emodin, rhein, chrysophanic acid anthrone, and 2,3,5,4′-tetrahydroxystilbene-2-O-β-D-glucoside, among others.

“Component of Lycium Barbarum” as used herein refers to any fraction, extract, or isolated or purified molecule from Lycium Barbarum. For example, the component is a protein or nucleic acid or a polysaccharide, a phytochemical, or a fraction of Lycium Barbarum. Thus, in certain embodiments of the invention, components of Lycium Barbarum are obtained by disrupting Lycium Barbarum, adding an inorganic or organic solvent, and collecting fractions. Specific, non-limiting examples of fractions are isolated using high performance liquid chromatography, thin layer chromatography, or distillation. Fractionation may be based on the molecular weight or the hydrophobicity of the components of Lycium Barbarum.

“Component of Aphanizomenon flos aquae” or “component of AFA” as used herein refers to any fraction, extract, or isolated or purified molecule from Aphanizomenon flos aquae. For example, the component is a protein or nucleic acid or a polysaccharide, a phytochemical, or a fraction of Aphanizomenon flos aquae. In another example, a carbohydrate-rich fraction can be derived by mechanical separation of particulate matter from the water-soluble fraction. A crude polysaccharide fraction can be obtained by extracting AFA for 4 hours with 70% ethanol at 65 degrees C., centrifuging ethanol extract, evaporating to dryness, resulting in a yield that is approximately 30% of AFA original dry weight.

“Differentiation” as used herein refers to the process by which cells become more specialized to perform biological functions. For example, hematopoietic stem cells, hematopoietic progenitors and/or stem cells may change from multipotent stem cells into cells committed to a specific lineage and/or cells having characteristic functions, such as mature somatic cells. Differentiation is a property that is often totally or partially lost by cells that have undergone malignant transformation.

“Enhancement,” “enhance” or “enhancing” as used herein refers to an improvement in the performance of or other physiologically beneficial increase in a particular parameter of a cell or organism. At times, enhancement of a phenomenon is quantified as a decrease in the measurements of a specific parameter. For example, migration of stem cells may be measured as a reduction in the number of stem cells circulating in the circulatory system, but this nonetheless may represent an enhancement in the migration of these cells to areas of the body where they may perform or facilitate a beneficial physiologic result, including, but not limited to, differentiating into cells that replace or correct lost or damaged function. In one embodiment, enhancement refers to a 15%, 20%, 30% or greater than 50% reduction in the number of circulating stem cells. In one specific, non-limiting example, enhancement of stem cell migration may result in or be measured by a decrease in a population of the cells of a non-hematopoietic lineage, such as a 15%, 20%, 30%, 50%, 75% or greater decrease in the population of cells or the response of the population of cells. In one embodiment, an enhanced parameter is the trafficking of stem cells. In one embodiment, the enhanced parameter is the release of stem cells from a tissue of origin. In one embodiment, an enhanced parameter is the migration of stem cells. In another embodiment, the parameter is the differentiation of stem cells. In yet another embodiment, the parameter is the homing of stem cells.

“Fucoidan” as used herein describes sulfated fucans obtained from algae. Fucoidan has been obtained from a broad range Algae species as provided in the following non-exhaustive list: Cladosiphon okamuranus, Chordaria flagelliformis, Ch. Gracilis, Saundersella simplex, Desmaestia intermedia, Dictyosiphon foeniculaceus, Dictyota dichotoma, Padina pavonica, Spatoglussum, schroederi, Adernocystis utricularis, Pylayella littoralis, Ascophyllum nodosum, Bifurcaria bifurcata, Fucus. Visculosus, F. spiralis, F. serratus, F. evaescens, Himanthalia lorea, Hizikia fusiforme, Pelvetia canaliculata, P. wrightii, Sargassum stenophyllum, S. honeri, S. Khellmanium, S. muticum, Alaria fistulosa, A. marginata, Arthrothammus bifidus, Chorda film, EckIonia kurome, E. cava, Eisenia bicyclis, Laminaria angustata, L. brasiliensis, L. cloustoni, L. digitata, L. japonica, L. religiosia, L. saccharina, Macrocystis integrifolia, M. pyrifera, Nereocystis luetkeana, Undaria pinnatifida, Petalonia fascia, Scytosiphon lomentaria. Substantial pharmaceutical research has been done on fucoidan, focusing primarily on two distinct forms: F-fucoidan, which is >95% composed of sulfated esters of fucose, and U-fucoidan, which is approximately 20% glucuronic acid, each of which is included in the term “fucoidan” as used herein. Depending on the source of the fucoidan, fucoidan can serve as a releasing agent in certain embodiments, while in other embodiments, fucoidan can serve as a migration agent.

“Hematopoiesis” as used herein refers to the formation and development of blood cells. Prenatally, hematopoiesis occurs in the yolk sack, then liver, and eventually the bone marrow. In normal adults, it occurs primarily in bone marrow and lymphatic tissues. All blood cells develop from pluripotent stem cells, which are committed to three, two, or one hematopoietic differentiation pathways. This includes the production of hematopoietic cells including B-cells, T-cells, cells of the monocyte macrophage lineage, and red blood cells.

“Hematopoietic agent” as used herein refers to a compound, antibody, nucleic acid molecule, protein, cell or other molecule that affects hematopoiesis. A molecular agent can be a naturally-occurring molecule or a synthetic molecule. In some instances, the agent affects the growth, proliferation, maturation, migration or differentiation or release of hematopoietic cells. In various embodiments, the agent is Lycium Barbarum, or an extract or component of Lycium Barbarum.

“Hematopoietic stem cells” as used in the present invention means multipotent stem cells that are capable of eventually differentiating into all blood cells including, erythrocytes, leukocytes, megakaryocytes, and platelets. This may involve an intermediate stage of differentiation into progenitor cells or blast cells. The term “hematopoietic progenitors”, “progenitor cells” or “blast cells” are used interchangeably in the present invention and describe maturing HSCs with reduced differentiation potential, but are still capable of maturing into different cells of a specific lineage, such as myeloid or lymphoid lineage. “Hematopoietic progenitors” include erythroid burst forming units, granulocyte, erythroid, macrophage, megakaryocyte colony forming units, granulocyte, erythroid, macrophage, and granulocyte macrophage colony-forming units.

“Homing” as used herein refers to the process of a cell migrating from the circulatory system into a tissue or organ. In some instances, homing is accomplished via tissue-specific adhesion molecules and adhesion processes. Homing may refer to the migration back to the bone marrow.

“Immunologically normal” as used herein refers to a subject that displays immune system characteristics typical for the species to which the individual belongs. These typical characteristics include, among others, functioning B-cells and T-cells as well as structural cell components, called cell surface antigens, which act as the immunologic signature for a particular organism.

“Immunologically compromised” as used herein refers to a subject having a genotypic or a phenotypic immunodeficiency. A genotypically-immunodeficient subject has a genetic defect that results in an inability to generate either humoral or cell-mediated responses. A specific, non-limiting example of a genotypically immunodeficient subject is a genotypically immunodeficient mouse, such as a SCID mouse or a bg/nu/xid mouse. A “phenotypically-immunodeficient subject” is a subject, which is genetically capable of generating an immune response, which has been phenotypically altered such that no response is seen. In one specific, non-limiting example, a phenotypically-immunodeficient recipient has been irradiated. In another specific, non-limiting example, a phenotypically-immunodeficient subject has been treated with chemotherapy. In yet another specific, non-limiting example, the phenotypically-immunodeficient subject has suffered a bacterial or viral infection, such as the human immunodeficiency virus (HIV) or simian immunodeficiency virus (SIV).

“Isolated biological component” (such as a nucleic acid molecule, polypeptide, polysaccharide or other biological molecule) as used herein refers to a biological component that has been substantially separated or purified away from other biological components in which the component naturally occurs. Nucleic acids and proteins may be isolated by standard purification methods, recombinant expression in a host cell, or chemically synthesized.

“Lycium Barbarum” or “L. Barbarum” as used herein refers to a small bright orange-red, ellipsoid berry or fruit grown. One exemplary source is in the north of China, primarily in the Ningxia Hui Autonomous Region. It is sometimes referred to as goji berry or wolfberry. L. Barbarum belongs to the Solanaceae family, the nightshade family that includes hundreds of plant foods like potato, tomato, eggplant, and peppers (paprika).

“Lymphoproliferation” as used herein refers to an increase in the production of lymphocytes.

“Modulation” or “modulates” or “modulating” as used herein refers to upregulation (i.e., activation or stimulation), down regulation (i.e., inhibition or suppression) of a response or the two in combination or apart.

“Migration” as used herein refers to the central process for movement of cells in the development and maintenance of multicellular organisms. Cells often migrate in response to, and towards, specific external signals, commonly referred to as chemotaxis. Migration includes the process of a cell moving from the circulatory system into a tissue or organ. More specifically, circulating stem cells are tethered to the surface of capillary endothelium via expression of adhesion molecules of cell surfaces, resulting in cytoskeletal changes in both endothelium and stem cells, and allowing movement through the capillary wall en route to a tissue and/or organ site. In some instances, homing is accomplished via tissue-specific adhesion molecules and adhesion processes.

“Migration agent” as used herein are mobilization agents capable of promoting the process of a cell moving from the circulatory system into a tissue or organ. Migration of stem cells may be demonstrated, for example, by a decrease in circulating stem cells in the circulatory or immune system, or by the expression of surface markers and/or adhesion molecules on cell surfaces, which relate to homing, tethering, and/or extravasation of circulating stem cells to the surface of vessels such as capillary endothelium. Examples of migration agents include isolated or purified components extracted from Aphanizomenon flos aquae, including a polysaccharide-rich fraction (fraction A) and a water soluble fraction (fraction B), Lycium Barbarum, including a polysaccharide-rich fraction (fraction A) of Lycium Barbarum extract, colostrum, including a protein-rich fraction (fraction B) of colostrum extract, fucoidan, including an isolated component or compound extracted from an algae, such as a compound found in a polysaccharide-rich fraction (fraction C) of algae extracts, including Chordaria cladosiphon, or other algaes, or extracts thereof, mushrooms, including an isolated component or compound extracted from a mushroom, such as a compound found in a polysaccharide-rich fraction (fraction D) of mushroom extracts, including Cordyceps sinensis or an extract thereof, Ganoderma lucidum or an extract thereof, Hericium erinaceus or an extract thereof, spirulina, including Arthrospira platensis, Arthrospira maxima, or extracts thereof. In different embodiments, this agent affects the migration of stem cells, such as CD34^{high} (CD34+) cells. In another embodiment, this agent affects the circulation of activated and/or quiescent VSELs. In one embodiment, the migration agent decreases the number of bone marrow-derived stem cells and/or hematopoietic stem cells circulating in the peripheral blood. In another embodiment, the migration agent relates to enhanced expression of CXCR4 on circulating stem cells.

“Mushroom polysaccharides” as used herein refers to glucans found mainly in various species of mushrooms such as Cordyceps sinesis, Hercicium erinaceous, and Ganoderma lucidum. This also includes the numerous bioactive polysaccharides or polysaccharide-protein complexes from medicinal mushrooms that may enhance innate and cell-mediated immune responses, and exhibit antitumor activities in animals and humans.

“Pharmaceutically acceptable carriers” as used herein refer to conventional pharmaceutically acceptable carriers useful in this invention.

“Polygonum multiflorum” or “P. multiflorum”, as used herein, refers to a species of herbaceous perennial vine growing to 2-4 m tall from a woody tuber native to central and southern China. Leaves are 3-7 cm long and 2-5 cm broad, broad arrowhead-shaped, with an entire margin. Flowers are 6-7 mm diameter, white or greenish-white, produced on short, dense panicles up to 10-20 cm long. Fruit is an achene 2.5-3 mm long. It is also known as Fallopia multiflora, Radix Polygoni, Radix Polygoni Multiflori, fleeceflower, He Shou Wu, or Fo-Ti.

“Polysaccharide” as used herein refers to a polymer of more than about ten monosaccharide residues linked glycosidically in branched or unbranched chains.

“Progenitor cell” as used herein refers to a cell that gives rise to progeny in a defined cell lineage.

“Promote” and/or “promoting” as used herein refer to an augmentation in a particular behavior of a cell or organism. In one embodiment, promoting relates to the mobilization of melanocyte derived stem cells. In another embodiment, promoting relates to the differentiation of stem cells into melanocytes.

“Recruitment” of a stem cell as used herein refers to a process whereby a stem cell in the circulatory system migrates into specific site within a tissue or organ. Recruitment may be facilitated by a compound or molecule, such as a chemoattractant signal or cell receptor. For example, both CXCR4 and SDF-1 have identified roles in stem cell homing and migration.

“Releasing agent” as used herein are mobilization agents capable of promoting the release and egress of stem cells from a tissue of origin. Release of stem cells from a tissue of origin may be demonstrated, for example, by an increase in circulating stem cells in the circulatory or immune system, or by the expression of markers related to egress of stem cells from a tissue of origin, such as bone marrow. Examples of releasing agents include fucoidan, as obtained from an extract of algae such as Undaria pinnatifida. In one embodiment, the releasing agent increases the number of bone marrow-derived stem cells and/or hematopoietic stem cells in the peripheral blood. In another embodiment, the releasing agent affects the number of stem cells, such as CD34^{high} (CD34+) cells, circulating in the peripheral blood. In another embodiment, the releasing agent affects the number of circulating activated and/or quiescent VSELs in the peripheral blood of a subject.

“Satellite cell” as used herein refers to a muscle-specific stem cell, often located in the periphery of muscle tissue, and capable of migrating into a muscle to aid in tissue repair and reconstruction.

“Stem cells” as used herein are cells that are not terminally differentiated and are therefore able to produce cells of other types. Characteristic of stem cells is the potential to develop into mature cells that have particular shapes and specialized functions, such as heart cells, skin cells, or nerve cells. Stem cells are divided into three types, including totipotent, pluripotent, and multipotent. “Totipotent stem cells” can grow and differentiate into any cell in the body and thus, can form the cells and tissues of an entire organism. “Pluripotent stem cells” are capable of self-renewal and differentiation into more than one cell or tissue type. “Multipotent stem cells” are clonal cells that are capable of self-renewal, as well as differentiation into adult cell or tissue types. Multipotent stem cell differentiation may involve an intermediate stage of differentiation into progenitor cells or blast cells of reduced differentiation potential, but are still capable of maturing into different cells of a specific lineage. The term “stem cells”, as used herein, refers to totipotent, pluripotent stem cells and multipotent stem cells capable of self-renewal and differentiation. “Bone marrow-derived stem cells” are primitive stem cells found in the bone marrow which can reconstitute the hematopoietic system, and possess endothelial, mesenchymal, and pluripotent capabilities. Stem cells may reside in the bone marrow, either as an adherent stromal cell type, or as a more differentiated cell that expresses CD34, either on the cell surface or in a manner where the cell is negative for cell surface CD34. “Adult stem cells” are a population of stem cells found in adult organisms with some potential for self-renewal and are capable of differentiation into multiple cell types. Specific examples of stem cells are marrow stromal cells (MSCs), hematopoietic stem cells (HSGs), marrow isolated adult multilineage inducible (MIAMI) cells, multipotent adult progenitor cells (MAPCs), very small embryonic-like stem cells (VSELs), epiblast-like stem cell (ELSCs) or primitive blastomere-like stem cell (BLSCs).

“Stem cell circulation agent” (SCCA), “mobilization agent”, and/or “mobilization factor” as used herein refers to one or more compounds, antibodies, nucleic acid molecules, proteins, polysaccharides, cells, or other molecules, including, but not limited to, neuropeptides and other signaling molecules, that affects the release, circulation, homing and/or migration of stem cells from the circulatory system into tissue or organ. A molecular agent may be a naturally occurring molecule or a synthetic molecule. Examples of mobilization agents include “releasing agents”, wherein a releasing agent is capable of promoting the egress of stem cells from a tissue of origin and also “migration agents”, wherein a migration agent is capable of promoting the process of a cell moving from the circulatory system into a tissue or organ.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

“Therapeutically effective amount” as used herein refers to the quantity of a specified composition, or active agent in the composition, sufficient to achieve a desired effect in a subject being treated. For example, this can be the amount effective for enhancing migration of stem cells that replenish, repair, or rejuvenate tissue. In another embodiment, a “therapeutically effective amount” is an amount effective for enhancing trafficking of stem cells, such as increasing release of stem cells, as can be demonstrated by elevated levels of circulating stem cells in the bloodstream. In still another embodiment, the “therapeutically effective amount” is an amount effective for enhancing homing and migration of stem cells from the circulatory system to various tissues or organs, as can be demonstrated be decreased level of circulating stem cells in the bloodstream and/or expression of surface markers related to homing and migration. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

“Trafficking” as used herein refers to the process of movement of a cell from the tissue of origin, traveling within the circulatory or immune system, and localization towards a site within a tissue and/or organ. Trafficking also includes stem cell mobilization, beginning with release from a tissue of origin, such as egress of stem cells from bone marrow. Trafficking further includes movement of a cell from the tissue of origin, homing by adhesion to the endothelium, transmigration, and final migration within the target tissue and/or organ. Furthermore, trafficking may include the process of movement of a cell of the immune system. One specific, non-limiting example of trafficking is the movement of a stem cell to a target organ, also referred to as migration. Another specific, non-limiting example of trafficking is the movement of a B-cell or a pre-B-cell leaving the bone marrow and moving to a target organ.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

As described, adult stem cells (ASCs) are a heterogeneous population, with different members varying in stem cells in plasticity (i.e., differentiation potential) and self-renewal capacity. One categorization provides four generic types of ASCs, as first presented in order of increasing plasticity and self-renewal capacity: 1) germ layer lineage stem cells, 2) progenitor cells, 3) epiblast-like stem cells, and 4) blastomere-like stem cells.

It is known that classic adult stem cells, such as germ layer lineage hematopoietic stem cells (HSCs) and bone marrow stem cells (BMSCs), animate the healing and regenerative processes of hematopoietic and immune systems of the body. This is accomplished through movement of HSCs and BMSCs from storage compartments in the body, and towards sites of regeneration or repair. For example, a key source of HSCs and BMSCs is bone marrow, which includes hip, ribs, sternum and other bone structures. Bone marrow is a unique regulatory microenvironment for HSCs and BMSCs, with extracellular matrix glycoproteins and a rich mineral signature. These features provide a “niche” with critical molecular interactions that guide the response of stem cells towards specific physiological conditions.

Movement of HSCs and BMSCs out of the niche leads to appearance of these cells in the peripheral bloodstream of normal, healthy persons. Through circulation in the bloodstream, HSCs and BMSCs move towards sites of maintenance and/or repair through combinations of receptors expressed on the cell surface (e.g., CXCR4), and sites expressing chemoattractant, Stromal-Derived Factor-1 (SDF-1). (Drapeau, 2010).

Among newly discovered actors within the four generic types of ASCs, it is of great interest to understand if their participation in regeneration and repair processes mirrors that of previously identified ASCs, such as HSCs and BMSCs. This includes understanding the role of very small embryonic-like (VSEL) stem cells, which may represent some of the most primitive ASCs found in an adult organism. These VSEL cells may possess near totipotency and or pluripotency akin to that of embryonic stem cells (ESCs). As such, they may be categorized as the most primitive type of ASCs, blastomere-like stem cells (embryonic blastomeres are one source of ESCs). The functional similarity of VSELs to ESCs, such as wide plasticity, partially accounts for the nomenclature of very small embryonic-like (VSEL) stem cells. A second aspect accounting for the nomenclature of VSEL is their very small size (sometimes less than <1-2 μm diameter) compared to other cells.

VSELs have been described as possessing near totipotency based on formation of virtually all somatic cell types, in addition to spermatagonia (Young and Black, 2005b). This capability of developing into cells derived from all three germ layers (endoderm, mesoderm, ectoderm) is remarkably similar to the pluripotency hallmark of ESCs (Young, J., et al. 2005). Notably, these cells also express markers characteristic of ESCs (e.g., Oct-4, Nanog, Rex-1, among others) (Zuba-Surma, et al. 2009). Beyond similarities in marker expression, VSELs also appear similar to ESCs based on observations of a euchromatic (“open chromation”) nuclear state similar to ESCs, and karyotypic stability.

It is further known that VSELs are incredibly rare (a few as 0.01% of the total population of bone marrow mononuclear cells) (Id.) VSEL cells may be descendants of stem cells present during early gastrulation/organogenesis that survive into adulthood. Upon further development, VSELs mature into slightly more committed epiblast-like stem cells (ELSCs), which cannot form non-somatic tissues (e.g., gametes), but are still capable of forming all three germ layers. ELSCs are also larger (often between <6-8 μm diameter), which then given rise to multipotent progenitor cells, and finally germ layer specific stem cells. Beyond VSELs, other types of multipotent cells, such as multilineage inducible (MIAMI) cells, have also been recently identified. These cells, also possess a wide differentiation potential across three germ layers and high proliferation rate, although their larger size (>7 μm), may categorize them as ELSCs or a newly discovered type of multipotent progenitor cells. (D'Ippolito et al., 2004).

In view of their wide differential potential and robust self-renewal capacity, VSELs may provide a significant and unique contribution to healing and regeneration across all the tissues and organs in the body. Similar to their HSC and BMSC counterparts, VSELs express CXCR4 and respond to chemokine, SDF-1. This suggests their capability to move out of stem cell niches, leading to appearance of VSELs in the peripheral bloodstream of normal, healthy persons. Indeed, most recent studies have identified VSELs as present within peripheral blood (Kucia, Stem Cells, abstract). In addition, those results further indicated that VSELs are responsive to mobilization agents that promote movement of HSCs and BMSCs out of the niche, and towards sites of maintenance and repair. This includes, as one example, application of granulocyte colony-stimulating factor (G-CSF), to increase numbers of VSELs in peripheral blood. (Id.)

Mobilization agents (also known as stem cell circulation agents, or mobilization factors) function, in part, by manipulating the mechanical and chemoattractant signals by which stem cells circulate in the peripheral bloodstream and are recruited to sites of tissue in need of repair and regeneration. For example, mechanical force or other factors may activate L-selectins on the surface of stem cells. Activation of L-selectins, in turn, may promote elevated expression of the receptor, CXCR4. Cells at the site of tissue injury may also secrete SDF-1 ligand, thereby attracting stem cells expressing receptor CXCR4 to the injury site. The interaction of SDF-1 and CXCR4 promotes sufficient adhesion to halt circulation of a stem cell in the peripheral blood stream. (Drapeau, 2010). Previously, the inventors have demonstrated a variety of mobilization agents as capable of promoting the trafficking of HSCs and BMSCs. Examples include U.S. Pat. Nos. 7,651,690, 8,034,328, and PCT Pub. No. WO 2012/006100. The expression of CXCR4 by VSELs, and responsiveness to SDF-1 strongly suggests these mobilization agents as capable of promoting similar effects on VSELs.

In addition to tapping into a newly discovered and potentially potent resource of VSELs for regeneration and repair, the inventors' mobilization agents provide other further benefits. Existing methods of promoting stem cell mobilization suffer from significant drawbacks, including poor kinetic performance, high cost, inconvenient methods of administration and unwanted side effects. Granulocyte colony-stimulating factor (G-CSF) or recombinant forms thereof, requires days to achieve peak circulating HSC numbers. The opposite problem exists with administration of interleukin-8 (IL-8), which acts only within minutes and has a short-lived effect on elevating circulating HSC levels in the bloodstream. (Frenette et al., 2000; Jensen et al., 2007) G-CSF and a different molecule, CXCR4 antagonist AMD3100, can have significant side effects, including hemorrhaging, rupturing of the spleen, bloody sputum, bone disorders, among others. Thus, there is a need in the art for an effective and convenient method for delivering stem cell mobilization agents to human subjects, to obtain positive clinical benefits without side effects and at a reduced cost.

Polygonum multiflorum.

The dried root tuber of Polygonum multiflorum plant, also known as fleeceflower root, has been used as a traditional Chinese medicine called He shou wu, this medication gaining notoriety in TCM from a tale of a famous Chinese military officer condemned to death and jailed without food or drink. Surviving by consuming the leaves and roots of the vinelike weed, Polygonum multiflorum, the officer's captors later found his remains as still having lustrous black hair. While the origins of this tale are apocryphal, they serve to illustrate the long-held notion that Polygonum multiflorum possesses important properties for tapping into the regenerative and restorative potential of the body. Recent scientific studies have confirmed that extracts of Polygonum multiflorum are indeed capable of promoting hair follicle growth, through increased expression of sonic hedgehog (Shh) and β-catenin expression—two important pathways involved in both early embryogenesis and maintaining stem cell identity. (Park et al. 2011)

Further analysis of Polygonum multiflorum extracts have confirmed this plant to be a rich source of bioactive compounds, two notable examples being anthraquinones and derivatives and hydroxyy stilbenes. Anthraquinones and derivatives have served as the basis for antimalarial, laxative, and chemotherapy treatments. Hydroxyl stilbenes, such as 2,3,5,4′-tetrahydroxystilbene-2-O-β-D-glucoside, have been show to provide important neuroprotective effects warding off symptoms of different neurodegenerative diseases. Together, these results indicate that components of Polygonum multiflorum extracts possess important properties for healing and regenerating the body, possibly by modulating inflammation, reducing risk of cancer proliferation, and/or providing protective effects for cells, tissues, and organs of the body.

While effects of these components in Polygonum multiflorum is somewhat understood for certain specific conditions, there is much less knowledge about how components of Polygonum multiflorum may specifically influence stem cell activity in the body. This is surprising given that, as described, stem cells play an integral role in the body's natural healing and regeneration mechanisms. One of the few existing studies on the subject indicates that Polygonum multiflorum extracts promotes proliferation of stem cells and progenitors, as shown by an increase in the number of bone marrow stem cells and lymphoid progenitors following administration of Polygonum multiflorum extracts in mice. (Zhiweng et al. 1991). Similarly, U.S. patent application Ser. No. 12/006,221 describes an increase in GM-CSF and stem cell factor (SCF) expression following administration in mice. These results present intriguing questions about potential effects of Polygonum multiflorum extracts on stem cell activity, given that both GM-CSF and SCF are implicated as playing important roles in stem cell migration and mobilization, as described above.

Fucoidan.

Fucoidan (also known as fucoidin or fucansulfate in the art) is a sulfated fucan polysaccharide L-selectin agonist that was documented to promote the egress of HSCs from compartments in bone marrow into the peripheral blood stream upon intravenous injection, although this effect seemed unrelated to its stimulation of L-selectin (Frenette et al., 2000). Circulation of HSCs in the peripheral bloodstream is a critical step in promoting the stem cell regeneration and repair mechanisms in the body. As a sulfated fucan, fucoidan is found in various species of algae. Other sulfated fucans have also been found in animal species, such as echinoderms (e.g., sea urchins and sea cucumbers).

As fucoidan is a sulfated fucose polysaccharide L-selectin ligand, its selectin activity depends on important carbohydrate or polypeptide modifications such as sialylation, fucosylation, and sulfation. The presence of binding sites for sulfated fucans such as fucoidan on P- and L-Selectin has been demonstrated to be at least partially the mechanism by which fucoidan promotes detachment of HSCs from BM. (Frenette et al., 2000, 2461, Jensen et al., 2007, 190) Perhaps more significantly, sulfated fucans such as fucoidan, have been shown to displace SDF-1 sequestered on endothelial surfaces or bone marrow through completive binding to a heparin-binding domain present on SDF-1. Occupation of the heparin-binding site of SDF-1 by fucoidan prevents tethering to cell surfaces, thereby increasing circulating SDF-1 levels in plasma. (Sweeney et al., 2008)

Without being bound by any particular theory, the enhanced levels of SDF-1 ligand in the bloodstream may thus promote egress of CXCR4 receptor expressing VSELs from the bonw marrow. Based on this model, the inventors hypothesized that L-selectin ligands, such as fucoidan, may possess a critical capacity to mobilize VSELs and oral administration of dietary supplements composed of fucoidan may best support natural regeneration and repair in the body.

The present invention provides new compositions and methods for providing a wide range of clinical and physiological benefits to a subject in need thereof by the administration of a mobilization agent. While not wishing to be bound by any particular theory, the inventors believe that the beneficial and other physiological results obtained through administration of the inventive compositions result from enhancing stem cell trafficking and migration that follows the administration of the mobilization agent.

In various embodiments, the mobilization agent comprises one or more components selected from the group including: blue-green algae (e.g., Aphanizomenon flos aquae), Polygonum multiflorum, Lycium Barabrum, colostrum, mushroom polysaccharides (e.g., Cordyceps sinensis, Hericium erinaceus (Lion's mane), Ganoderma lucidum (Reishi)), fucoidan (optionally extracted from algaes, e.g., Undaria pinnatifida, Chordaria cladosiphon (Limu)), spirulina (e.g., Arthrospira platensis, Arthrospira maxima), analogs thereof, derivatives thereof, extracts thereof, synthetic or pharmaceutical equivalents thereof, fractions thereof, and combinations of any of the foregoing items.

The mobilization agents may be combined together in one or more compositions or they may be administered or consumed separately as part of a regimen. They may have individual physiological effects, additive effects and/or synergistic effects with one another, such as serving as both a releasing agent and migration agent. In some embodiments, the mobilization agent is capable of functioning as a migration agent, promoting the process of a cell moving from the circulatory system into a tissue or organ. In some embodiments, the mobilization agent is capable of functioning as a releasing agent, promoting the release and egress of stem cells from a tissue of origin. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In one embodiment, a mobilization agent is administered to a subject, for example a blue-green algae, such as Aphanizomenon flos aquae (AFA), though the subject may be provided a mixture of blue-green algae and other mobilization agents. In some embodiments, the subject consumes and digests whole blue-green algae. Blue-green algae may be fresh, frozen, freeze-dried, dehydrated, or preserved in some other manner. In one embodiment, the mobilization agent is an extract of blue-green algae, or an isolated component or compound extracted from blue-green algae, such as a compound found in a polysaccharide-rich fraction of blue-green algae extract, or a compound in a water soluble compartment of an blue-green algae extract. Blue-green algae can be provided alone as an isolated or purified substance, or may be part of a composition including a pharmaceutically acceptable carrier. In one embodiment, blue-green algae is capable of functioning as a migration agent. In one embodiment, blue-green algae is capable of functioning as a releasing agent. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In one embodiment, a mobilization agent is administered to a subject, for example Polygonum multiflorum, though the subject may be provided a mixture of Polygonum multiflorum, and other mobilization agents. In some embodiments, the subject consumes and digests whole Polygonum multiflorum root, leaves, stem, seeds, fruits, and/or other plant parts. The whole Polygonum multiflorum root, leaves, stem, seeds, fruits, and/or other plant parts may be fresh, frozen, freeze-dried, dehydrated, fermented, or preserved in some other manner. Therefore, Polygonum multiflorum, as described herein, encompasses whole Polygonum multiflorum root, leaves, stem, seeds, fruits, and/or other plant parts. In other embodiments, the mobilization agent is an extract of Polygonum multiflorum, or an isolated component or compound extracted from Polygonum multiflorum, such as a compound found in a polysaccharide-rich fraction of Polygonum multiflorum extracts, or a fraction soluble in aqueous solutions, or a fraction soluble in organic solvents. Polygonum multiflorum can be provided alone as an isolated or purified substance, or may be part of a composition including a pharmaceutically acceptable carrier. In one embodiment, Polygonum multiflorum or extracts thereof is capable of functioning as a migration agent. In one embodiment, Polygonum multiflorum or extracts thereof is capable of functioning as a releasing agent. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

Extracts of components found in Polygonum multiflorum include anthraquinones and derivatives, hydroxyl siltbenes, lecithin, chrysophanol, chrysophanic acid, chrysophanol anthrone, emodin, physcion, rhein, chrysophanic acid anthrone, resveratrol, piceid, 2,3,5,4′-tetrahydroxystilbene-2-O-β-D-glucopyranoside, 2,3,5,4′-tetrahydroxystilbene-2-O-β-D-glucopyranoside-2″-O-monogalloyl ester, 2,3,5,4′-tetrahydroxystilbene-2-O-β-D-glucopyranoside-3″-O-monogalloyl ester, 2,3,5,4′-tetrahydroxystilbene-2-O-β-D-glucoside, gallic acid, catechin, epicatechin, 3-O-galloyl(−)-catechin, 3-O-galloyl(−)-epicatechin, 3-O-galloyl-procyanidin B-2,3,3′-di-O-galloyl-procyanidin B-2, and β-sitosterol.

The identity and nature (e.g., stability) of components in prepared Polygonum multiflorum extracts may vary depending on the method used for extraction. For example, water extraction is a leading method of exacting components from Polygonum multiflorum. However, certain components, such as anthraquinones and derivatives are very insoluble in water. Anthraquinones and derivatives are also insoluble in organic solvents at room temperature, but soluble in hot organic solvents (e.g., boiling temperature), such as methanol or ethanol. Similarly, 2,3,5,4′-tetrahydroxystilbene-2-O-β-d-glycoside is known to readily degrade in aqueous solutions in a temperature and pH dependent manner. (Ren et al. 2011) Therefore, it is understood that extracts of Polygonum multiflorum may be prepared according to any method known in the art. This includes, water extraction, organic solvent extraction (e.g., U.S. patent application Ser. No. 12/006,221), or combinations of such exemplary methods (e.g., admixtures). Examples of organic solvents that may be used include methanol, n-hexane, ethyl acetate, and n-butanol. Combinations of two or more water and/or organic solvents could be added together to generate additional partition layers for extracting different components in different partition layers. Similarly, extracts from Polygonum multiflorum may be prepared from fresh, unprocessed whole plants or parts thereof, or extracts may be prepared from processed Polygonum multiflorum whole plants or parts thereof. For example, processing may be performed by any known method in the art, one example being fermentation. Processing may improve bioavailability of the components in extracts from Polygonum multiflorum, such as through fermentation with bacteria such as Lactobacillus sp. (Park et al., 2011) or through addition of black beans.

In one embodiment, a mobilization agent is administered to a subject, for example Lycium Barbarum, though the subject may be provided a mixture of Lycium Barbarum and other mobilization agents. In some embodiments, the subject consumes and digests whole Lycium Barbarum berries. The berries may be fresh, frozen, freeze-dried, dehydrated, or preserved in some other manner. Therefore, Lycium Barbarum, as described herein, encompasses both whole berry and extracts thereof. In one embodiment, the mobilization agent is an extract of Lycium Barbarum, or an isolated component or compound extracted from Lycium Barbarum, such as a compound found in a polysaccharide-rich fraction of Lycium Barbarum extract. Lycium Barbarum can be provided alone as an isolated or purified substance, or may be part of a composition including a pharmaceutically acceptable carrier. In one embodiment, Lycium Barbarum is capable of functioning as a migration agent. In one embodiment, Lycium Barbarum is capable of functioning as a releasing agent. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In one embodiment, colostrum is administered to a subject, though the subject may be provided a mixture of colostrum and other mobilization agents. In some embodiments, the subject consumes and digests whole colostrum. The colostrum may be fresh, frozen, freeze-dried, dehydrated, or preserved in some other manner. Therefore, colostrum, as described herein, encompasses both whole colostrum and extracts thereof. In one embodiment, the mobilization agent is an extract of colostrum, or an isolated component or compound extracted from colostrum, such as a compound found in a protein-rich fraction of colostrum extract colostrum can be provided alone as an isolated or purified substance, or may be part of a composition including a pharmaceutically acceptable carrier. In one embodiment, colostrum is capable of functioning as a migration agent. In one embodiment, colostrum is capable of functioning as a releasing agent. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In one embodiment, mushroom or a blend of mushrooms is administered to a subject, though the subject may be provided a mixture of mushrooms and other mobilization agents. In some embodiments, the subject consumes and digests whole mushrooms. The mushrooms may be fresh, frozen, freeze-dried, dehydrated, or preserved in some other manner. Therefore, mushrooms, as described herein, encompass both whole mushrooms and extracts thereof. In one embodiment, the agent is Cordyceps sinensis or an extract thereof. In one embodiment, the mobilization agent is Ganoderma lucidum or an extract thereof. In one embodiment, the mobilization agent is Hericium erinaceus or an extract thereof. Mushrooms can be provided alone as isolated or purified substances, or may be part of a composition including a pharmaceutically acceptable carrier. In one embodiment, mushrooms, Cordyceps sinensis, Ganoderma lucidum, and/or Hericium erinaceus is capable of functioning as a migration agent. In one embodiment, mushrooms, Cordyceps sinensis, Ganoderma lucidum, and/or Hericium erinaceus is capable of functioning as a releasing agent. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In one embodiment, algae is administered to a subject, though the subject may be provided a mixture of algae and other mobilization agents. In some embodiments, the subject consumes and digests whole algae. The algae may be fresh, frozen, freeze-dried, dehydrated, or preserved in some other manner. Therefore, algae, as described herein, encompass both whole mushrooms and extracts thereof. In one embodiment, the mobilization agent is Chordaria cladosiphon or an extract thereof. Algae can be provided alone as isolated or purified substances, or may be part of a composition including a pharmaceutically acceptable carrier. In one embodiment, algae, Chordaria cladosiphon is capable of functioning as a migration agent. In one embodiment, algae, Chordaria cladosiphon is capable of functioning as a releasing agent. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In one embodiment, spirulina is administered to a subject, though the subject may be provided a mixture of spirulina and other mobilization agents. In some embodiments, the subject consumes and digests whole spirulina. The spirulina may be fresh, frozen, freeze-dried, dehydrated, or preserved in some other manner.

Therefore, spirulina, as described herein, encompasses both whole spirulina and extracts thereof. In one embodiment, the mobilization agent is Arthrospira platensis, Arthrospira maxima, or an extract thereof. Spirulina can be provided alone as an isolated or purified substance, or may be part of a composition including a pharmaceutically acceptable carrier. In one embodiment, spirulina is capable of functioning as a migration agent. In one embodiment, spirulina is capable of functioning as a releasing agent. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

The present invention further provides a method of enhancing the trafficking of stem cells in a subject. In one embodiment, the level of trafficking of stem cells relates to the number of circulating CD34+ stem cells in the peripheral blood of a subject. In another embodiment, the level of trafficking of stem cells relates to the number of circulating activated and/or quiescent VSELs in the peripheral blood of a subject. In another embodiment, the level of trafficking of stem cells relates to the number of circulating stem cells in the peripheral blood of a subject. In different embodiments, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

A method is described herein for enhancing stem trafficking by administering to a subject a therapeutically effective amount of blue-green algae.

In one embodiment, blue-green algae, such as Aphanizomenon flos aquae (AFA) is administered to a subject, though the subject may be provided a mixture of more than one algae. In some embodiments, the subject consumes and digests whole algae. The blue-green algae, may be fresh, frozen, freeze-dried, dehydrated, or preserved in some other manner.

In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In alternative embodiments, an extract of blue-green algae, such as AFA, is provided or administered to the subject. In another embodiment, blue-green algae, encompasses both whole plant, parts of the plant, and/or extracts thereof. In another embodiment, the blue-green algae can be provided alone as an isolated or purified substance, or may be part of a composition including a pharmaceutically acceptable carrier. In another embodiment, the extract is water soluble compartment. In another embodiment, the extract is a polysaccharide rich compartment.

A method is described herein for enhancing stem trafficking by administering to a subject a therapeutically effective amount of Polygonum multiflorum.

In one embodiment, Polygonum multiflorum, is administered to a subject, though the subject may be provided a mixture of more than one ingredient. In some embodiments, the subject consumes and digests whole plant or parts of the plant. The Polygonum multiflorum may be fresh, frozen, freeze-dried, dehydrated, or preserved in some other manner.

In alternative embodiments, an extract of Polygonum multiflorum is provided or administered to the subject. In another embodiment, the Polygonum multiflorum encompasses both whole plant, parts of the plant, and extracts thereof. In another embodiment, the Polygonum multiflorum can be provided alone as an isolated or purified substance, or may be part of a composition including a pharmaceutically acceptable carrier. In another embodiment, the extract is an anthraquinone and/or derivative. In another embodiment, the extract is a hydroxylstilbene. In an alternative embodiment, whole Polygonum multiflorum plant is administered to the subject. In another embodiment, parts of Polygonum multiflorum plant are administered to the subject. In one embodiment, an extract of Polygonum multiflorum is administered to the subject.

A method is described herein for enhancing stem trafficking by administering to a subject a therapeutically effective amount of fucoidan.

In one embodiment, an algae, such as Undaria pinnatifida, is administered to a subject, though the subject may be provided a mixture of more than one algae. In some embodiments, the subject consumes and digests whole algae. The algae may be fresh, frozen, freeze-dried, dehydrated, or preserved in some other manner.

In alternative embodiments, an extract of the algae is provided or administered to the subject. In another embodiment, the algae encompasses both whole plant and/or extracts thereof. In another embodiment, the algae can be provided alone as an isolated or purified substance, or may be part of a composition including a pharmaceutically acceptable carrier. In another embodiment, the extract is a highly sulfated, polyanionic soluble fiber. In one embodiment, the extract is an isolated fucoidan. In a different embodiment, the fucoidan is purified following isolation. In an alternative embodiment, a polysaccharide fraction is administered to the subject. In another embodiment, the highly sulfated, polyanionic soluble fiber is administered to the subject. In one, the isolated fucoidan is administered to the subject. In a different embodiment, the purified fucoidan is administered to the subject. In one embodiment, Undaria pinnatifida is capable of functioning as a releasing agent after administration to a subject.

The present invention further provides a method of enhancing the trafficking of stem cells in a subject. In one embodiment, the level of trafficking of stem cells relates to the number of circulating CD34+ stem cells in the peripheral blood of a subject. In another embodiment, the level of trafficking of stem cells relates to the number of circulating activated and/or quiescent VSELs in the peripheral blood of a subject. In another embodiment, the method provided herein enhances the trafficking of stem cells in a subject, including administering a therapeutically effective amount of a composition containing one or more of the following components selected from the group including: blue-green algae, such as Aphanizomenon flos aquae, or extracts thereof, Polygonum multiflorum or extracts thereof, Lycium barbarum or extracts thereof, colostrum or extracts thereof, spirulina or extracts thereof, Arthrospira platensis or extracts thereof, Arthrospira maxima or extracts thereof, fucoidan, Chordaria cladosiphon or extracts thereof, Hericium erinaceus or extracts thereof, Ganoderma Lucidum or extracts thereof, and/or Cordyceps Sinensis or extracts thereof, thereby enhancing the trafficking of stem cells in the subject. In one embodiment, enhancement of stem cell trafficking may be measured by assaying the response of stem cells to a particular dose of a composition containing one or more of the following components selected from the group including: blue-green algae, such as Aphanizomenon flos aquae, or extracts thereof, Polygonum multiflorum or extracts thereof, Lycium barbarum or extracts thereof, blue-green algae or extracts thereof, colostrum or extracts thereof, spirulina or extracts thereof, Arthrospira platensis or extracts thereof, Arthrospira maxima or extracts thereof, fucoidan or extracts thereof, Chordaria cladosiphon or extracts thereof, Hericium erinaceus or extracts thereof, Ganoderma Lucidum or extracts thereof, and/or Cordyceps Sinensis or extracts thereof, thereby enhancing the trafficking of stem cells in the subject.

In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

The present invention further provides a method of reducing inflammation in a subject. In another embodiment, the method provided herein reduces inflammation in a subject, including administering a therapeutically effective amount of a composition containing one or more of the following components selected from the group including: blue-green algae, such as Aphanizomenon flos aquae, or extracts thereof, Polygonum multiflorum or extracts thereof, Lycium barbarum or extracts thereof, colostrum or extracts thereof, spirulina or extracts thereof, Arthrospira platensis or extracts thereof, Arthrospira maxima or extracts thereof, fucoidan, Chordaria cladosiphon or extracts thereof, Hericium erinaceus or extracts thereof, Ganoderma Lucidum or extracts thereof, and/or Cordyceps Sinensis or extracts thereof, thereby enhancing the trafficking of stem cells in the subject. In one embodiment, the level of inflammation relates to fibrogenesis of stem cells. In one embodiment, the fibrogenesis is modulated by levels of platelet derived growth factor. In one embodiment, the mobilization agent does not activate platelets.

In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell. In one embodiment, the mobilization agent does not activate platelets and reduces fibrogenesis of VSELs.

The present invention further provides a pharmaceutical preparation. In one embodiment, the pharmaceutical preparation is 90%, 80%, 70%, 60%, 50%, 40%, 30%, 20%, 10%, 5%, or 1% w/w Aphanizomenon flos aquae (AFA) or extracts thereof. In one embodiment, the pharmaceutical preparation is 90%, 80%, 70%, 60%, 50%, 40%, 30%, 20%, 10%, 5%, or 1% w/w Polygonum multiflorum. In one embodiment, the pharmaceutical preparation is 90%, 80%, 70%, 60%, 50%, 40%, 30%, 20%, 10%, 5%, or 1% w/w fucoidan.

The present invention further provides a dosing regimen. In one embodiment, the dosing regimen is dependent on the severity and responsiveness of a disease state to be treated, with the course of treatment lasting from a single administration to repeated administration over several days and/or weeks. In another embodiment, the dosing schedule is based on measurement of an active component accumulated in the body. In a certain embodiment, the active component is fucoidan. In one embodiment, the fucoidan is isolated from Undaria pinnatifida or extracts thereof. In another embodiment, the dosing regimen is dependent on the level of stem cell trafficking in the subject. In one embodiment, the dosing regimen is dependent on the activity of a releasing agent administered to a subject. In another embodiment, the dosing regimen is dependent on the number of circulating CD34+ stem cells in the peripheral blood stream of a subject. In another embodiment, the dosing regimen is dependent on the number of circulating activated and/or quiescent VSELs in the peripheral blood of a subject. In another embodiment, the dosing regimen is dependent on the number of circulating bone marrow-derived stem cells in the peripheral blood stream of a subject. In one embodiment, the dosing regimen is 3 grams of fucoidan administered daily. In another embodiment, the dosing regimen is 1 gram of fucoidan administered daily. In another embodiment, the dosing regimen is 500 mg grams of fucoidan administered daily. In another embodiment, the dosing regimen is 75 mg grams of fucoidan administered daily. In one embodiment, the dosing regimen is 250 mg grams of fucoidan administered daily.

The present invention further provides a method for enhancing the trafficking of stem cells in a subject, comprising administering a therapeutically effective amount of a mobilization agent or a polysaccharide fraction of a mobilization agent, thereby increasing the release, circulation, homing and/or migration of stem cells in the subject, regardless of the route of administration.

The present invention further provides a method of inducing a transient increase in the population of circulating stem cells, such as CD34+ stem cells, following administration of a mobilization agent. In another embodiment, the transient increase relates to the number of circulating activated and/or quiescent VSELs in the peripheral blood of a subject. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In one embodiment, enhancement of stem cell trafficking may be measured by assaying the response of stem cells to a particular dose of mobilization agent. In one embodiment, providing the mobilization agent to a subject will enhance release of that subject's stem cells within a certain time period, such as less than 12 days, less than 6 days, less than 3 days, less than 2, or less than 1 days. In an alternative embodiment, the time period is less than 12 hours, 6 hours, less than about 4 hours, less than about 2 hours, or less than about 1 hour following administration.

In one embodiment, administration of a mobilization agent results in the release of stem cells into the circulation from about 2 to about 3 hours following administration. In another embodiment, released stem cells enter the circulatory system and increase the number of circulating stem cells within the subject's body. In another embodiment, the percentage increase in the number of circulating stem cells compared to a normal baseline may about 25%, about 50%, about 100% or greater than about 100% increase as compared to a control. In one embodiment, the control is a base line value from the same subject. In another embodiment, the control is the number of circulating stem cells in an untreated subject, or in a subject treated with a placebo or a pharmacological carrier.

The present invention further provides of a method of inducing a transient decrease in the population of circulating stem cells, such as CD34+ stem cells. In another embodiment, the transient decrease relates to the number of circulating activated and/or quiescent VSELs in the peripheral blood of a subject. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In embodiment, enhancement of stem cell migration may be measured by assaying the response of stem cells to a particular dose of mobilization agent. In one embodiment, providing a mobilization agent to a subject will enhance migration of that subject's stem cells within a certain time period, such as less than about 5 hours, less than about 4 hours, less than about 2 hours, or less than about 1 hour following administration.

In another embodiment, the percentage decrease in the number of circulating stem cells compared to a normal baseline may about 25%, about 50%, about 100% or greater than about 100% increase as compared to a control. In one embodiment, the control is a base line value from the same subject. In another embodiment, the control is the number of circulating stem cells in an untreated subject, or in a subject treated with a placebo or a pharmacological carrier.

In another embodiment, administration of an extract of algae increases the rate of homing of stem cells measured by a transient decrease in the number of circulating stem cells within the subject's body. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In another embodiment, the algae is Chordaria cladosiphon. In another embodiment, the percentage decrease in the number of circulating stem cells compared to a normal baseline may about 25%, about 50%, about 75%, or even about 100% as compared to a control. In one embodiment, the control is a base line value from the same subject. In another embodiment, the control is the number of circulating stem cells in an untreated subject, or in a subject treated with a placebo or a pharmacological carrier.

In one embodiment, administration of a mobilization agent results in the migration of stem cells from the circulation to tissues from about 1 to about 3 hours following administration. Circulating stem cells will leave the circulatory system, thus decreasing the number of circulating stem cells within the subject's body. The percentage decrease in the number of circulating stem cells compared to a normal baseline may be about 15%, about 30%, about 50% or greater than about 75% decrease as compared to a control. In one embodiment, the control is a base line value from the same subject. In another embodiment, the control is the number of circulating stem cells in an untreated subject, or in a subject treated with a placebo or a pharmacological carrier. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In another embodiment, administration of an extract of a mobilization agent increases the rate of homing of stem cells measured by a transient decrease in the number of circulating stem cells within the subject's body. The percentage decrease in the number of circulating stem cells compared to a normal baseline may be about 25%, about 50%, about 75%, or even about 100% as compared to a control. In one embodiment, the control is a base line value from the same subject. In another embodiment, the control is the number of circulating stem cells in an untreated subject, or in a subject treated with a placebo or a pharmacological carrier. In another embodiment, the administration of an extract of a mobilization agent leads to an increase in CXCR4 expression on circulating stem cells. In one embodiment, the stem cell is a germ layer lineage stem cell, progenitor cell, epiblast-like stem cell (ELSC) or blastomere-like stem cell (BLSC). In one embodiment, the stem cell is a very small embryonic-like (VSEL) stem cell.

In some embodiments, the subject administered a mobilization agent is healthy. In other embodiments, the subject is suffering from a disease or physiological condition, such as immunosuppression, chronic illness, traumatic injury, degenerative disease, infection, or combinations thereof. In certain embodiments, the subject may suffer from a disease or condition of the skin, digestive system, nervous system, lymph system, cardiovascular system, endocrine system, or combinations thereof. In specific embodiments, the subject suffers from osteoporosis, Alzheimer's disease, cardiac infarction, Parkinson's disease, traumatic brain injury, multiple sclerosis, cirrhosis of the liver, any of the diseases and conditions described in the Examples below, or combinations thereof. Administration of a therapeutically effective amount of a mobilization agent may prevent, treat and/or lessen the severity of or otherwise provide a beneficial clinical benefit with respect to any of the aforementioned conditions, although the application of the inventive methods and use of the inventive mobilization agent is not limited to these uses. In various embodiments, the novel compositions and methods find therapeutic utility in the treatment of, among other things, skeletal tissues such as bone, cartilage, tendon and ligament, as well as degenerative diseases, such as Parkinson's and diabetes. Enhancing the release, circulation, homing and/or migration of stem cells from the blood to the tissues may lead to more efficient delivery of stem cells to a defect site for increased repair efficiency. The novel compositions and methods of the present invention may also be used in connection with gene therapeutic approaches.

The present invention further provides various compositions for administration to a subject. In one embodiment, the administration is topical, including ophthalmic, vaginal, rectal, intranasal, epidermal, and transdermal. In one embodiment, the administration is oral. In one embodiment, the composition for oral administration includes powders, granules, suspensions or solutions in water or non-aqueous media, capsule, sachets, tablets, lozenges, or effervescents. In another embodiment, the composition for oral administration further comprises thickeners, flavoring agents, diluents, emulsifiers, dispersing aids or binding agents.

Described herein are mobilization agents and methods of using mobilization agents towards promoting stem cell trafficking. Further described herein are migration agents and method of using migration agents to promote the process of stem cells moving from the circulatory system into a tissue or organ. Also described herein are releasing agents and methods of using releasing agents to promote egress of stem cells from a tissue of origin. The inventors have demonstrated effective administration of stem cell mobilization agents, thereby achieving a safe, convenient and effective method to enhance stem cell-related maintenance and repair in the human body. Although the pathology of stem cells is of great importance and interest, and pertains to the subject matter disclosed herein, the underlying scope of this invention is that the release, circulation, homing and/or migration of stem cells from the blood to tissues is of significance in repairing injured tissue and maintaining the vitality and health of existing tissue. Thus, the importance of developing methods and compositions for achieving this end are among the foci and aims of the present invention.

Accordingly, the present invention provides novel compositions and methods for, among other things, enhancing natural tissue healing and renewal in the body by supporting the trafficking of stem cells. Furthermore, the present invention provides novel compositions and methods for preventing, slowing or otherwise diminishing the development of health problems in a mammal by promoting trafficking of stem cells in the mammal. The compositions and methods disclosed herein may further increase regeneration of existing tissue by supporting the release, circulation, homing and/or migration of stem cells into tissue, therefore supporting the process of tissue repair.

### EXAMPLES

The following examples are provided to better illustrate the claimed invention and are not to be interpreted as limiting the scope of the subject matter. To the extent that specific materials are mentioned, it is merely for purposes of illustration and is not intended to limit the invention. One skilled in the art may develop equivalent means, compositions or reactants without the exercise of inventive capacity and without departing from the scope of the present invention.

### Example 1

### Study Design: Mobilization of Human VSELs

Preliminary experiments on healthy human subjects as well as equines indicate that ingestion of AFA increase the total number of VSELs circulating in the peripheral blood of mammals. The inventors conducted a study on 35 adult, healthy human volunteers between the ages of 20 years to 70 years old. Prior to entering the study, the subjects signed informed consent forms under an IRB approved by Mercer University School of Medicine. The entry criteria for the study was that the subjects were healthy, not on prescription medication, and tested serologically negative against a panel of infectious agents such as HIV, Hepatitis C virus, HTLV, cytomegaloviruses and STDs.

To determine the effects of AFA on the mobilization of human VSELs into peripheral circulation, blood was drawn by venous puncture from human subjects and collected into vacutainer tubes containing 12 mg of 15% ethylenediaminetetraacetic acid (EDTA).

This initial blood draw served as control samples in which the plasma was fractionated and enriched for VSELs. Venous puncture and isolation of VSELs was performed as follows. Following puncture and blood withdraw according to standard medical procedure, blood was placed in tubes, that were inverted 3-4×, to disperse EDTA solution into mixture. Blood collection tubes were then placed in 4° C. refrigeration for 48 hours. Following 48 hours of gravity separation, blood is separated into a plasma-rich fraction, and cellular fraction containing red/white blood cells, hematopoietic stem cells and other types of stem cells. The plasma fraction is removed, and stored in a separate tube at 4° C. Fifteen microliters of this solution was mixed with 15 microliters of sterile 0.4% Tyrpan blue, and placed on hemacytometer. VSELs are Trypan blue positive and <2.0 microns in size. Slight more committed VSELs, such as those transitioning to epiblast-like stem cells, are Trypan blue positive or negative, and are 3-5 microns in size. Epiblast like stem cells will not stain for Trypan blue and are over 6 microns in size. Additional examples of methods for purifying for VSELs may be found, for example, in U.S. Pat. Pub. No. 2009/0104158.

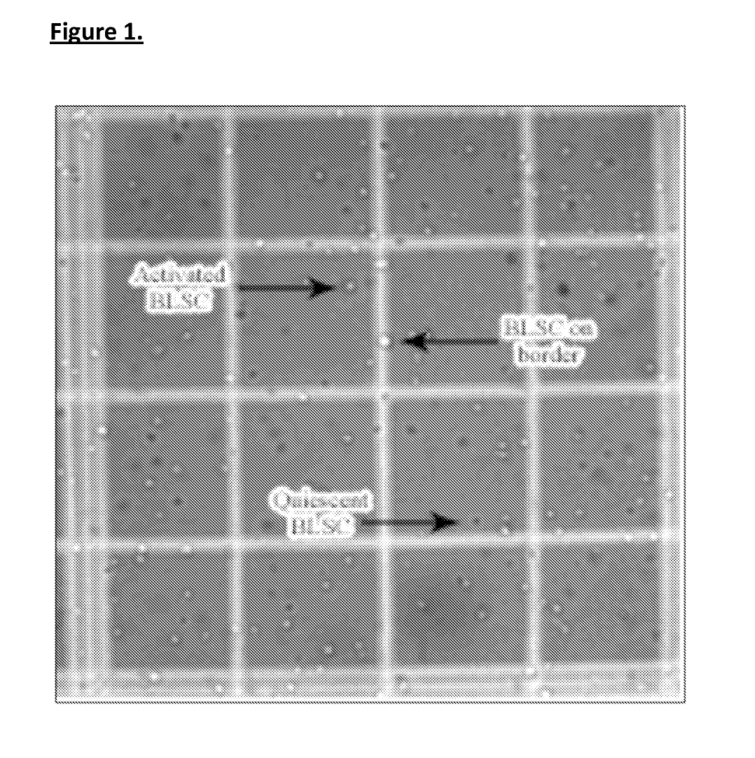

With a hemacytometer, cell counts were performed manually from an aliquot of the subject's fractionated plasma in order to determine the number of both quiescent and activated VSELs (see FIG. 1). At the time of the initial blood draw, cell counts from the control samples served as the baseline for calculating the percentage increase in the number of both quiescent and activated VSELs circulating in the subject's fractionated plasma. Immediately after the initial blood draw, the subjects ingested two capsules of StemEnhance. A second blood draw was performed on the same subject one hour after StemEnhance ingestion. Cell counts were again conducted to determine the effects of AFA on the number of VSELs mobilized into peripheral circulation.

### Example 2

### Assay of Quiescent and Activated VSELs

Additionally, activated VSELs were determined microscopically by their: i) increased size (>2 μm); ii) reduced vital dye uptake; and, iii) their light refractive properties. Additionally, quiescent VSELs are defined as small microcells (<2 μm), non-refractive to light, and having an inability to exclude the vital dye, Trypan Blue (see FIG. 1). An analysis of the mobilization results revealed that AFA ingestion increases the total amount of circulating VSELs by an average of 32.44% with a standard deviation of 0.64 (see Table 1). Median percentage increase of the total amount of VSELs in circulation is 18.37%. It was found that 1 hr. after AFA ingestion there is approximately a 20-30% increase in the number of VSELs circulating in the blood.

TABLE 1

| | Median % Increase | Average % Increase | Standard |
|---|---|---|---|
| | after AFA ingestion | after AFA ingestion: | Deviation: |
| Total Cell | 18.37% | 32.44% | 0.6432 |
| Number | | | |
| Activated | 21.21% | 19.33% | 0.2742 |
| VSELs | | | |
| Quiescent | 8.75% | 50.52% | 2.2299 |
| VSELs | | | |

### Example 3

### Mobilization Agents for VSELs

In assaying for the two VSEL subpopulations (i.e. activated and quiescent VSELs), the percent increase in the total number of activated VSEL in the blood after AFA ingestion was 19.33%. Similarly, the percent increase of quiescent VSELs was 50.52%. Median percent increase of activated VSELs was 21.21° A, while the median percent increase of quiescent VSELs was 8.75%. The variation in the cell counts for the two subpopulations among the subjects may be attributed to several factors such as the age and physiological state of a subject. For example, these differences between subjects can lead to the large variation between the median percent increase and the average percent increase observed in the quiescent VSEL subpopulation. Depending on the physiological state of a subject at the time of the blood draw, the number of quiescent VSELs being mobilized may vary within the same individual. Nevertheless, most of the 35 subjects involved in the study showed both an increase in quiescent and activated VSELs circulating in the blood after AFA ingestion. It should be also noted that outliers in the study fell into one of three different categories listed below:
1. an increase in quiescent VSELs and a decrease in activated VSELs.
2. a decrease in quiescent VSELs coupled with an increase in activated VSELs.
3. a decrease in both activated and quiescent VSELs.

### Example 4

### Activation of Human VSELs

Analyzing activation of human VSELs was determined by a change in their morphology. Microscopic analyses with Trypan blue as a vital stain were performed for analyzing the cells under direct light. Wet mounts under phase contrast microscopy supported the measurements from the cell counts with the counting chambers. Activated VSELs are described as being Trypan blue negative, bright in color on a neutral background, round, and roughly 2 μm and larger in size. Quiescent VSELs are Trypan blue positive, dark in color on a neutral background, round in morphology, and roughly 1-2 μm in size. These two subpopulations of VSELs were counted separately, before and after AFA ingestion. Both the median and average percent increase for each subpopulation were calculated from the compiled data.

The results from the cell count analyses using the above parameters showed that the average percent increase after AFA ingestion of activated VSELs was 19.33%. Median percent increase after AFA ingestion was 21.21%. The cell count data showed that AFA ingestion consistently results in a 19-22% increase of activated VSELs with the exceptions of a few outliers.

The outliers found in this part of the study for assaying activated VSELs in the blood of a particular subject may also be attributed to variables confounding the results such as age, health, stress levels, and other physiological differences between subjects. Similar outliers were also observed in measuring the quiescent VSEL subpopulation in the blood of a subject.

### Example 5

### In Vitro Analysis of Human VSEL

Similar to the in vivo mobilization study, VSELs were harvested from the subjects' blood and seeded onto tissue culture plates. Blood samples were obtained prior to ingestion of AFA (T0) and one hour after ingesting two capsules of AFA (T1). The rationale for these experiments was to determine if the physiological effects of AFA could be assayed in vitro. The blood was drawn into 7 mL vacutainer tubes containing EDTA and processed at various time intervals for VSEL enriched fractionated plasma. The VSEL plasma fraction (VSEL-pf) was harvested from the blood samples 24 hours, 48 hours, and 72 hours after the initial blood draw. These three time intervals were used to determine if AFA ingestion enhanced VSEL activation in vitro and induced the release of putative stem cell growth factors by other cellular constituents in the blood samples.

After cell counts were performed, each sample was seeded at a density of 1.0×10⁶ cells/cm2 in tissue culture media. (Serum-Free Defined BLSC Basal Medium, pH 7.4: prepared with 5 mL of Antibiotetic-Antimycotic solution and 495 mL of BLSC Basal Medium, catalog #MBC-ASB-MED-100-A002, Moraga Biotechnology Corporation). Further culturing of VSELs can be performed according to techniques known in the art. This include, for example, methods described in U.S. Pat. Pub. No. 2009/0104160.

Additionally, the samples were seeded at different dilutions in order to dissect whether post-ingestion of AFA could affect the VSEL cultures with respect to: i) adherence; ii) increased numbers of refractile granules; iii) formation of spheroid bodies; and, iv) formation of fibrin matrices. Blood drawn from four healthy subjects ranging in age from 45 to 52 years old (who were serologically negative for a panel of infectious agents) were used for assaying in vitro VSEL-pf.

Two modified protocols for harvesting and processing the VSEL-pf were used for the in vitro study. One procedure, “Process 1,” was developed in which harvesting the VSEL-pf excluded platelet activation and thus, the platelet-derived growth factor (PDGF) in the enriched plasma fraction containing the VSELs should be at lower levels. As an example, “Process 1” relies on double-centrifugation for platelet removal. Blood collected in centrifuge capped citrate tube(s), were first spun for 10 minutes at an RCF (relative centrifugal force) of 1500-2000 g. The top ¾ of plasma fraction was removed using plastic transfer pipette, without disturbance of buffy coat or cell fraction. This separate plasma fraction was placed in a plastic centrifuge tube, and spun for an additional 10 minutes at RCF 1500-2000 g. The top ¾ of this double-centrifugated plasma fraction was removed as a platelet poor plasma (PPP) fraction.

The second procedure, “Process 2,” incorporated standard protocol for harvesting the VSEL-pf in which platelets are activated and PDGF concentrations were expected be higher in the fractionated plasma using EDTA and gravity separation. As described above, this process relies on addition of EDTA solution into blood drawn from venous puncture, 48 hours of 4° C. refrigeration, and gravity separation for separation into the plasma-rich fraction and the cellular fraction containing red/white blood cells, hematopoietic stem cells and other types of stem cells.

### Example 6

### Results

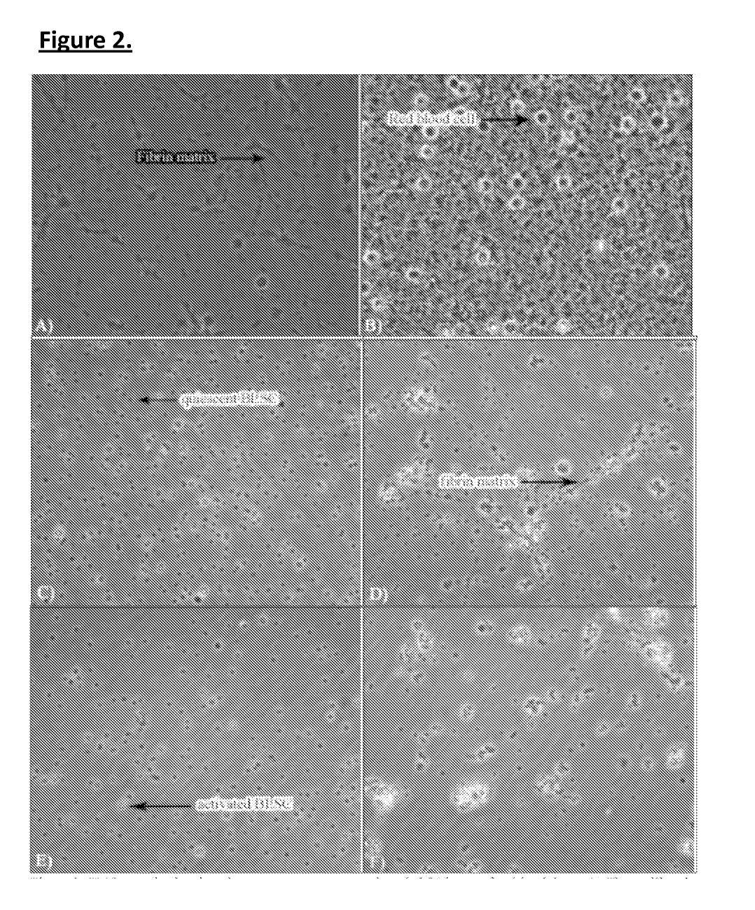

Preliminary in vitro results demonstrated that fibrin matrix formation (FIG. 2) occurred in undiluted VSEL-pf for both T0 and T1 samples. In these undiluted samples fibrin matrices were observed within 24 hour after seeding the VSEL-pf. It was difficult to discern if AFA ingestion affected fibrinogenesis in the undiluted samples. However, when the harvested plasma fractions were serially diluted, the time for observing fibrinogenesis occurred at a faster rate in the T0 samples than in the T1 samples. The cultures seeded at a 1:10 dilution showed the largest difference in the time fibrin matrix formation was initially observed between T0 and T1 cultures (Table II.). This observation suggested that AFA ingestion affected in vitro the rate kinetics for observing fibrinogenesis.

In order to determine whether AFA or its metabolites affected the rate for in vitro fibrin matrix formation, VSEL-pf were processed and cultured at different time intervals (i.e. days 1, 2, & 3) from the subject's blood. Table II is a summary of the experimental results reflecting the inhibitory effects of AFA ingestion which showed the amount of time required for observing fibrin matrix formation in the cell cultures.

TABLE 2
Average Days to Fibrin Matrix Formation

| | |
|---|---|
| Process 1 | Process 2 |

| | T₀ | T₁ | T₀ | T₁ |
|---|---|---|---|---|
| Day 1 Seeding | 4.2 | 5.5 | 3 | 4.75 |
| Day 2 Seeding | 3.25 | 2.25 | 2.5 | 2.5 |
| Day 3 Seeding | 2 | 2.25 | 2.5 | 2.5 |

### Example 7

### Effect of Mobilization Agent on Fibrogenesis in Plasma Serum Cultures

When the samples were harvested and the VSEL-pf processed one day after the blood draw, fibrin matrix formation was observed in the cultures 3-5 days after seeding the samples. More importantly, fibrinogenesis in the post-AFA ingestion samples (T1) required an additional 24 hrs. of incubation compared to the T0 cultures (blood samples harvested prior to ingesting AFA); suggesting an inhibitory effect on in vitro fibrinogenesis post-AFA ingestion. This in vitro effect appears to dissipate over time (shortening the time to form fibrin matrices) when comparing the time to form fibrin matrices in cultures seeded on either day 2 or day 3.

The in vitro experimental results also suggest that PDGF in the plasma fraction may reduce some of the anti-fibrinogeneic effect of AFA. The masking effect exhibited by PDGF may explain the rate differences observed during fibrin matrix formation in cultures using Process 1 compared to cultures seeded using Process 2 (PDGF enrichment protocol). It appears that the plasma fractions used for seeding the cultures on day 1 had a lower concentration of PDGF than the day 2 and day 3 samples. Thus, harvesting VSEL-pf with the Process 2 protocol may block some of the anti-fibrinogeneic in vitro effect following AFA ingestion.

### Example 8

### Analysis of Platelet-Derived Growth Factor in the Plasma

In order to confirm the presence of PDGF in the in vitro assays for fibrinogenesis ELISAs (Enzyme-Linked ImmunoSorbent assay) were conducted to interrogate the harvested human plasma for PDGF-BB (platelet-derived growth factor). With aliquots from VSEL-pf, ELISAs were performed on both T0 and T1 samples as well as from samples using Process 1 & 2.

### Example 9

### Methods

Ninety-six-welled plates were coated with a monoclonal anti-human PDGF-B subunit antibody and incubated overnight at room temperature. The plates were then washed with a solution of PBS-Tween (phosphate buffered saline plus Tween-20) and blocked with a buffer consisting of 1% BSA (bovine serum albumin) and PBS. The blocking buffer was then removed and the plates were washed again. After washing, serial dilutions of standard PDGF-BB and plasma were added to the plates. Standard PDGF-BB was used as a positive control. No detection antibody and no antigen were used as the negative controls in the assays. Both the standard PDGF and the plasma used for analysis were serial diluted 10-fold per dilution. The detection antibody was a biotinylated anti-PDGF-BB antibody. Following coating of the detection antibody, streptavidin alkaline-phosphatase was added to the plates followed by pnitrophenyl phosphate. The plates were then allowed to develop for approximately 30 minutes, washed and read with a plate reader at 405 nm absorbance. A standard titration curves were performed in order to calculate the concentration of the PDGF in the plasma (see table 2). The plasmas analyzed for PDGF were then compared before and after AFA ingestion (refer to section III for details). Plasma extracted before AFA ingestion is labeled as T0. Plasma extracted 1 hour after AFA ingestion is labeled as T1. Six subjects were used for this portion of the study.

### Example 10

### Results

Table III. summarizes the data obtained from the ELISAs. It should be noted that the concentrations of PDGF in the plasma samples generally corresponded with the number of VSELs harvested from the enriched plasma. Higher cellular concentration of VSELs in the plasma correlated with increased levels of PDGF in same plasma fraction.

TABLE 3

PDGF in the Plasma (T₀ vs. T₁)

| | Average | Standard |
|---|---|---|
| | Concentration (ng/ml) | Deviation |
| T₀ | 7.23 | 0.2720 |
| T₁ | 7.21 | 0.2729 |
| Difference (T₀ − T₁) | 0.015 | n/a |

The concentration of PDGF in the plasma ranged from 3.33 ng/ml to 10.2 ng/ml. The average concentration for PDGF for T0 was 7.23 ng/ml. For T1, the average PDGF concentration was 7.21 ng/ml. Overall, the concentration of PDGF in the plasma remained relatively constant before and after AFA ingestion as shown by the small difference (0.015 ng/ml) in the concentration of PDGF between T0 and T1.

TABLE 4
PDGF in the Plasma (Process 1 vs. Process 2)

| T₀ Process 1 | T₁ Process 1 | T₀ Process 2 | T₁ Process 2 |
|---|---|---|---|
| 4.34 ng/ml | 4.35 ng/ml | 5.52 ng/ml | 6.98 ng/ml |

Table IV. summarizes the concentration of PDGF in the harvested plasma fraction at the time seeding VSELs-pf into culture plates. ELISAs conducted on plasma fractions were harvested by two different methods (Process 1 or Process 2) appeared to affect the concentration of PDGF in the VSEL-pf. There was approximately a 20% increase in PDGF released into the enriched plasma fraction using the protocol in Example 1 for harvesting VSEL from a subject's blood. Additionally, AFA ingestion did not increase the plasma levels of PDGF.

### Example 11

### Effect of Mobilization Agents on VSEL Trafficking and Anti-Inflammatory Properties

The effects of StemEnhance (AFA) ingestion on mobilization and activation of Moraga's VSELs in the blood were demonstrated both in vivo and in vitro. An increase in the number of VSELs circulating in the blood was confirmed by conducting cell counts from a subject's blood prior to and after ingesting AFA. Interestingly, intense physical activity/stress (30 mins. on a treadmill) can also cause an increase in the number of circulating VSEL in the subject's blood. Additionally, StemEnhance ingestion also increases the number of activated VSELs circulating in the subject's blood. However, in vitro experiments as well as ELISA analyses suggest that AFA ingestion does not activate platelets. In vitro experiments revealed anti-inflammatory activity after AFA ingestion. Since an increase in plasma levels of fibrinogen is associated with inflammation, the results of the study showed the rate at which fibrinogen is converted into fibrin is decreased in the VSEL cultures following AFA ingestion. The in vitro results also unveil a potential role for PDGF in affecting the rate in which the formation of the fibrin matrix is initially observed in the cell cultures. The study results demonstrated conclusively that ingesting StemEnhance had the following effect:
1. AFA ingestion increases total cell number by 32.44%.
2. AFA ingestion increases activated VSELs by 19.33%.
3. AFA ingestion increases quiescent VSELs by 50.52%.
4. In vitro analyses suggest that AFA ingestion may have an anti-inflammatory effect.
5. The concentration of PDGF remains constant before and after AFA ingestion.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the sources of very small embryonic-like cells (VSELs), blastomere-like stem cells (BLSCs), epiblast-like stem cells (ELSCs), the methods of preparing, isolating, or purifying VSELs, BLSCs, ELSCs, stem cell mobilization agents, the methods of preparing, isolating, or purifying stem cell mobilization agents, analogs and derivatives thereof, methods of treating various disease and/or conditions using stem cell mobilization agents, analogs and derivatives thereof, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### REFERENCES

Abedi M., Greer D., Colvin G., Demers D., Dooner M., Harpel J., Pimentel J., Menon M., and Quesenberry P., Tissue injury in marrow transdifferentiation Blood Cells, Molecules and Diseases 2004 32, 42-46
Andriole G., Mulé J., Hansen C., Linehan W., Rosenberg S., Evidence that lymphokine-activated killer cells and natural killer cells are distinct based on an analysis of congenitally immunodeficient mice. Journal of Immunology 1985 135, 2911-2913.
Adams G. and Scadden D., The hematopoietic stem cell in its place. Nature Immunology 2006 7, 333-337.
Asahara T., Masuda H., Takahashi T., Kalka C., Pastore C., Silver M., Kearne M., Magner M., and Isner J., Bone Marrow Origin of Endothelial Progenitor Cells Responsible for Postnatal Vasculogenesis in Physiological and Pathological Neovascularization. Circulation Research 1999 85, 221-228.
Barker N, van de Wetering M, Clevers H. The intestinal stem cell. Genes and Development 2008 22, 1856-64.
Bertea O. and Mulloy B., Sulfated fucans, fresh perspectives: structures, functions, and biological properties of sulfated fucans and an overview of enzymes active toward this class of polysaccharide. Glycobiology 2003 13, 29-40.
Branski L., Gauglitz G., Herndon D., and Jeschke M., A review of gene and stem cell therapy in cutaneous wound healing. Burns, Jul. 4, 2008.
Burke Z., Thowfeequ S., Peran M., Tosh D. Stem cells in the adult pancreas and liver. Biochemistry Journal 2007 404, 169-78.
Dezawa M., Ishikawa H., Hoshino M., Itokazu Y., and Nabeshima Y., Potential of bone marrow stromal cells in applications for neuro-degenerative, neuro-traumatic and muscle degenerative diseases. Current Trends in Neuropharmacology 2005 3, 257-66.
D'Ippolito G., Diabira S., Howard G., Menei P., Roos B., Schiller P. Marrow-isolated adult multilineage inducible (MIAMI) cells, a unique population of postnatal young and old human cells with extensive expansion and differentiation potential. J Cell Sci. 2004 117, 2971-81.
Drapeau C., Cracking the Stem Cell Code, Sutton Hart Press (Portland, Oreg. 2010)
Fraser J., Schreiber R., Zuk P., and Hedrick M., Adult stem cell therapy for the heart. International Journal of Biochemistry & Cell Biology 2004 36, 658-666
Frenette P. and Weiss L., Sulfated glycans induce rapid hematopoietic progenitor cell mobilization: evidence for selectin-dependent and independent mechanisms. Hemostasis, Thrombosis, and Vascular Biology 2000 96, 2460-2468.
Hidalgo, A., Sanz-Rodriguez F., Rodriguez-Fernández J., Albella B., Blaya C., Wright N., Cabañas C., Prósper F., Gutierrez-Ramos J., Teixidó J., Chemokine stromal cell-derived factor-1 alpha modulates VLA-4 integrin-dependent adhesion to fibronectin and VCAM-1 on bone marrow hematopoietic progenitor cells. Experimental. Hematology, 2001 29, 345-55;
Irhimeh M., Fitton J., and Lowenthal R., Fucoidan ingestion increases the expression of CXCR4 on human CD34+ cells. Experimental Hematology 2007 35, 989-994.
Jang Y., Collector M., Baylin S., Diehl A., and Sharkis S., Hematopoietic stem cells convert into liver cells within days without fusion. Nature Cell Biology 2004, 6, 532-529.
Jensen, G. and Drapeau C., The use of in situ bone marrow stem cells for the treatment of various degenerative diseases. Medical Hypotheses 2002 59, 422-428.
Jensen, G., Hart A., Lue A., Drapeau C., Gupta N., Scaehffer D., and Cruickshank J. Mobilization of human CD34+CD133+ and CD34+CD133− stem cells in vivo by consumption of an extract from Aphanizomenon flose-aquae-related to modulation of CXCR4 expression by an L-selectin ligand? Cardiovascular Revascularization Medicine 2007 8, 189-202.
Kollet O., Spiegel A., Peled A., Petit I., Byk T., Hershkoviz R., Guetta E., Barkai G., Nagler A., Lapidot T., Rapid and efficient homing of human CD34(+)CD38(−/low)CXCR4(+) stem and progenitor cells to the bone marrow and spleen of NOD/SCID and NOD/SCID/B2m (null) mice. Blood 2001 97, 3283-91.
Krause D., Theise N., Collector M., Henegariu O., Hwang S., Gardner R., Neutzel S., and Sharkis S., Multi-organ, multi-lineage engraftment by a single bone marrow-derived stem cell Cell 2001 105, 369-377
Kuang S., Gillespie M., Rudnicki M. Niche regulation of muscle satellite cell self-renewal and differentiation. Cell Stem Cell 2008 10, 22-31.
Kucia M., Wysoczynski M., Wu W., Zuba-Surma E., Ratajczak J., Ratajczak M. Evidence that very small embryonic-like stem cells are mobilized into peripheral blood. Stem Cells. 2008 26, 2083-92.
McCune J., Namikawa R., Kaneshima H., Shultz L., Lieberman M., Weissman I., The SCID-hu mouse: murine model for the analysis of human hematolymphoid differentiation and function. Science 1998, 241, 1632-9.
Mourão, P., and Pereira, M., Searching for alternatives to heparin: sulfated fucans from marine invertebrates. Trends in Cardiovascular Medicine 1999 9, 225-232.
Park H., Zhanga N., Park D., Topical application of polygonum multiflorum extract induces hair growth of resting hair follicles through upregulating Shh and β-catenin expression in C57BL/6 mice. Journal of Ethnopharmacology. 2011 May 17; 135(2):369-75. Epub 2011 Mar. 17.
Pereira M., Melo F., and Mourao, P., Is there a correlation between structure and anticoagulant action of sulfated galactans and sulfated fucans? Glycobiology 2002 12, 573-580.
Ratajczak M., Zuba-Surma E., Wysoczynski M., Ratajczak J., Kucia M. Very small embryonic-like stem cells: characterization, developmental origin, and biological significance. Exp Hematol. 2008 36, 742-51.
Ren X., Wang G., Wang M., Ou-Yang H., Qi A. Kinetics and mechanism of 2,3,5,4′-tetrahydroxystilbene-2-O-β-d-glycoside (THSG) degradation in aqueous solutions. J Pharm Biomed Anal. 2011 Apr. 28; 55(1):211-5. Epub 2010 Dec. 31.
Revishchin A., Korochkin L., Okhotin V., Pavlova G. Neural stem cells in the mammalian brain. International Review Cytology. 2008 265, 55-109.
Stout C., Ashley D., Morgan J. 3rd, Long G., Collins J., Limnios J., Lochner F., McCommon G., Hixson D., Black A. Jr, Young H. Primitive stem cells residing in the skeletal muscle of adult pigs are mobilized into the peripheral blood after trauma. The American Surgeon. 2007 73, 1106-10.
Sweeney E., Priestly G., Nakamoto B., Collins R., and Beaudet A., Mobilization of stem/progenitor cells by sulfated polysaccharides does not require selectin presence. Proceedings of the National Academy of Sciences 2000 97, 6544-6549.
Sweeney E., Lortat-Jacob H., Priestly G., Nakamoto B. and Papayannopoulou T., Sulfated polysaccharides increase plasma levels of SDF-1 in monkeys and mice: involvement in mobilization of stem/progenitor cells. Blood 2008 99, 44-51.
Wang X., Foster M., Al-Dhalimy M., Lagasse E., Finegold M., and Grompe M. The origin and liver repopulating capacity of murine oval cells. Proceedings of the National Academy of Science 2003 Suppl. 1:11881-11888.
Yang L., Li S., Hatch H., Ahrens K., Cornelius J., Petersen B., Peck A. In vitro trans-differentiation of adult hepatic stem cells into pancreatic endocrine hormone-producing cells. Proceedings of the National Academy of Science 2002 99, 8078-83.
Young H., and Black A. Adults Stem Cells. Anat Rec A Discov Mol Cell Evol Biol. 2004 276, 75-102. (“Young 2004a”)
Young H., and Black A. Adult-derived stem cells. Minerva Biotechnologica. 2005 17, 55-63. (“Young 2005a”)
Young H., Duplaa C., Katz R., Thompson T., Hawkins K., Boev A., Henson N., Heaton M., Sood R., Ashley D., Stout C., Morgan J., Uchakin P., Rimando M., Long G., Thomas C., Yoon J., Park J., Hunt D., Walsh N., Davis J., Lightner J., Hutchings A., Murphy M., Boswell E., McAbee J., Gray B., Piskurich J., Blake L., Collins J., Moreau C., Hixson D., Bowyer F., Black A. Adult-derived stem cells and their potential for use in tissue repair and molecular medicine. J Cell Mol Med. 2005 9, 753-69. (“Young 2005b”)
Young H., Duplaa C., Romero-Ramos M., Chesselet M., Vourc'h P., Yost M., Ericson K., Terracio L., Asahara T., Masuda H., Tamura-Ninomiya S., Detmer K., Bray R., Steele T., Hixson D., el-Kalay M., Tobin B., Russ R., Horst M., Floyd J., Henson N., Hawkins K., Groom J., Parikh A., Blake L., Bland L., Thompson A., Kirincich A., Moreau C., Hudson J., Bowyer F., Lin T., Black A. Adult reserve stem cells and their potential for tissue engineering. Cell Biochem Biophys. 2004 40, 1-80. (“Young 2004b”)
Zuba-Surma E., Wu W., Ratajczak J., Kucia M., Ratajczak M. Very small embryonic-like stem cells in adult tissues-potential implications for aging. Mech Ageing Dev. 2009 130, 58-66.

1. A method of increasing stem cell mobilization in a subject, comprising:
providing a mobilization agent capable of increasing stem cell mobilization; and
administering a quantity of the mobilization agent to the subject in an amount sufficient to increase stem cell mobilization in the subject.

2. The method of claim 1, wherein the mobilization agent is a composition comprising one or more of the following components selected from the group consisting of: Aphanizomenon flos aquae or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma Lucidum or extracts thereof, and/or Cordyceps Sinensis or extracts thereof.

3. The method of claim 1, wherein the mobilization agent is Aphanizomenon flos aquae or extracts thereof.

4. The method of claim 1, wherein the mobilization agent is Polygonum multiflorum or extracts thereof.

5. The method of claim 1, wherein the mobilization agent is fucoidan.

6. The method of claim 1, wherein the stem cell is a very small embryonic-like (VSEL) stem cell.

7. The method of claim 6, wherein the VSEL cell is an activated or quiescent VSEL.

8. The method of claim 1, wherein the stem cell is a blastomere-like stem cell (BLSC) or epiblast-like stem cell (ELSC).

9. The method of claim 1, wherein administering the quantity comprises oral administration.

10. The method of claim 9, wherein the oral administration comprises use of a capsule or a pill.

11. A pharmaceutical composition comprising:
one or more of the following components selected from the group consisting of: Aphanizomenon flos aquae or extracts thereof, Polygonum multiflorum or extracts thereof, Lycium barbarum or extracts thereof, colostrum or extracts thereof, spirulina or extracts thereof, fucoidan, Hericium erinaceus or extracts thereof, Ganoderma Lucidum or extracts thereof, and/or Cordyceps Sinensis or extracts thereof; and
a pharmaceutically acceptable carrier.
